(19) United States
(12) Patent Application Publication
(10) Pub. No.: US 2023/0062797 A1
(43) Pub. Date: Mar. 2, 2023

(54) URINE ANALYSIS DEVICE AND METHOD

(71) Applicant: WITHINGS, ISSY LES MOULINEAUX (FR)

(72) Inventors: Thibaut BARBEDETTE, ISSY LES MOULINEAUX (FR); Jonas LOY, ISSY LES MOULINEAUX (FR); Marine KIRAT, ISSY LES MOULINEAUX (FR); Benoît TUCOULAT, ISSY LES MOULINEAUX (FR); Julius DEWAVRIN, ISSY LES MOULINEAUX (FR)

(21) Appl. No.: 17/799,599

(22) PCT Filed: Mar. 3, 2021
(86) PCT No.: PCT/EP2021/055377

§ 371 Date: Aug. 12, 2022

(30) Foreign Application Priority Data
Mar. 3, 2020 (FR) FR2002125

(51) Int. Cl. A61B 10/00; B01L 3/00; A61B 5/145; A61B 5/00; A61B 5/20

(52) U.S. Cl. CPC A61B 10/007; B01L 3/50; A61B 5/14507; A61B 5/6891; A61B 5/207

(57) ABSTRACT

A urine analysis device includes a case configured to be positioned entirely within a toilet, the case having a front face for receiving a stream of urine directly from a user urinating on the toilet a rear face opposite the front face, and a collection port, disposed on either the front face or the rear face, wherein the case contains a test assembly configured to perform an analysis on urine collected through the collection port.

### TECHNICAL AREA

The present disclosure is in the field of urine analysis devices to be positioned inside a toilet. The present disclosure also relates to a method for analyzing urine being received in a toilet.

### Previous Technique

Many biological parameters are reflected in the urine of an individual. For example, health problems such as urinary tract infection, diabetes or kidney failure can be detected from a urine sample. The urine sample can also reflect the quality of a diet, identify a fertile period or pregnancy and detect drug or tobacco use. It is then interesting to monitor various biological parameters periodically.

It is known to offer toilet devices with a urine analysis function. These devices are capable of taking urine samples from the toilet and analyzing them to determine the level of a biological parameter.

It is described in U.S. Pat. No. 5,720,054 and U.S. Ser. No. 10/383,606 to collect a urine sample through a mobile arm located in the toilet. However, this solution has the difficulty of collecting enough urine without air bubbles to perform a conclusive analysis. In addition, the use of a mobile arm adds complexity to the device.

A urine analysis device to be attached to a toilet rim is known from US20180188231. However, it appears that there is still room for improvement in urine collection.

US20170284925 proposes to integrate a urine sampler into the toilet bowl. Nevertheless, this solution requires adapting the toilet for the installation and use of the device.

Therefore, there is a need for a urine analysis device that can collect a urine sample from the toilet without the drawbacks of the prior art.

### Presentation of the Invention

The present description is intended to present urine analysis devices and associated methods that do not have at least one of the aforementioned drawbacks.

In particular, there is provided a urine analysis device comprising a case configured to be positioned entirely within a toilet, the case having a front face for receiving a stream of urine directly from a user urinating while seated on the toilet, a rear face opposite the front face, and a collection port, disposed on either the front face or the rear face, wherein the case contains a test assembly configured to perform an analysis on urine collected through the collection port.

Thus, advantageously, the urine analysis device can collect urine through the collection port directly by runoff on the sides of the case. A user does not need to worry about the position of the case when urinating in the toilet. In addition, the case is compact enough to be positioned entirely inside the toilet. It can be discreet and easily installed and removed. It can also be adapted to any type of toilet. In addition, the case has no protruding parts that are unpleasant for the user to see and that can collect dirt. The overall monobloc or monolithic appearance of the housing gives it an image of robustness and simplicity.

Urine collection is made easy and the user only has to perform a micturition without really worrying about the positioning of the device.

The features outlined in the following paragraphs can optionally be implemented. They can be implemented independently of each other or in combination with each other.

The collection port may be located near a lower end of the case in the normal use position. For example, the collection port may be located on a lower half of the case in the normal position of use. Thus, urine flowing by gravity down the sides of the case can be collected. The urine collection area is increased, it suffices that a small amount of urine reaches the housing to drip down the walls of the housing and reach the collection port.

The case may be deprived of ridges. Therefore, urine can run off the case without coming off or forming air bubbles.

The front face and/or rear face may include a relief forming a pathway for urine to the collection port. Wherever urine comes into contact with the case, it will be directed to the collection port. Thus, the urine collection surface is increased. The relief can be negative (like a recess) or positive (like a projection). The relief is formed in the case (front face and/or rear face), for example.

The collection port is located in a recess, having a border, the recess having two lateral grooves extending from an edge of the case to a central portion of the recess, the central portion of the recess housing the collection port. The depth of the lateral grooves may increase from the edge to the central portion. The border may have an inflection between at least one lateral groove and the central portion. The distance between the border of the recess and the edge of the case may increase from the beginning of the lateral groove to the central portion. The central part can have an area of between 3 and 8 cm².

The recess (or the central part of the recess) can extend towards the inside of the case over a distance of between 1 and 4 cm.

The case can have a general circular pebble shape. Thus, the shape of the case is defined by curved surfaces, and is a simple and natural shape that is pleasing to the eye of the user. Urine can run down the entire case without stalling or forming air bubbles.

The urine analysis device may have no moving parts outside the case. The urine analysis device does not require a seal. The case is free of stagnation points where urine could stagnate. The urine analysis device is hygienic.

The case may include a drain port configured to drain urine. Accordingly, excess urine collected may be purged from the urine analysis device. A urine collection is then not contaminated by a previous collection. Several consecutive urine collections can be performed.

The drain port and collection port can be equipped with a wire mesh filter. In this way, the filter can prevent the introduction of contaminants or elements that may clog the test system, and filter the urine before it reaches the collection port.

The case can be made of a hydrophilic material, preferably the hydrophilic material is one of: a ceramic, a polyamide, a silicone, a hydrophilic polymer, and/or the case can be treated with a hydrophilic surface treatment. Thus, urine coming into contact with the case will cling and spread on the case.

The diameter of the case can be between 50 mm and 150 mm, preferably the diameter can be between 80 mm and 120 mm, and even more preferably the diameter of the case is around 100 mm. The case is then sufficiently compact to be entirely received in the toilet. The case is discrete. The case is also large enough to systematically come into contact with urine being received in the toilet.

The case can be made of a front and a rear shell. The front and rear shells can be joined by screwing, gluing, clipping, magnetizing, or ultrasonic welding. So, the assembly of the case is easy. In addition, the joint between the front and rear shells can allow urine to flow between the front and rear shells.

The urine analysis device may include a sensor for the presence of urine, adjacent to the collection port, the sensor being configured to detect the presence of urine, preferably the sensor being a temperature sensor. Thus, the urine analysis device can initiate an analysis when urine is detected on the case.

The urine presence sensor can be a temperature sensor. The temperature sensor can distinguish between urine and water being detected on the case. In addition, the temperature sensor can detect a fertile period. Thus, the temperature sensor can both detect the presence of urine and perform an analysis. The number of components of the urine analysis device is reduced.

The urine analysis device may include a fastener configured to position the case on an inner wall of a toilet bowl. Then, the urine analysis device can be moved or removed from the toilet.

The urine analysis device may include a module for communication with a device and/or server and/or smartphone. Thus, the urine analysis device may be controlled to initiate an analysis. The urine analysis device may analyze and transmit one or more test results.

The device may include a button. The device may include a button, whereby an analysis can be triggered when a user presses the button.

The button can be equipped with a biometric sensor. This allows the user to be identified and the analysis is only triggered if a user is identified. An analysis can be adapted to the identified user.

The test assembly may comprise one or more of: a plurality of test strips, a micro-fluidic chip, a field effect transistor, a conductivity measuring device, a pH measuring device, a spectroscopic measuring device, an electrochemical measuring device. Thus, a single case can be adapted to analyze various biological parameters contained in urine. The urine analysis device is versatile.

According to another aspect, there is proposed a method of collecting urine operating the urine analysis device comprising:
detecting the presence of urine in the vicinity of the collection port;
collecting the urine running off the front face and/or rear face;
analyzing the collected urine to establish one or more analysis results.

The features outlined in the following paragraphs can optionally be implemented. They can be implemented independently of each other or in combination with each other.

The method can be triggered by an interaction with a user. The method can be triggered by pressing a button. The method can be initiated by detecting a user in the vicinity of the toilet. The method may also be initiated upon detection of urine on the urine analysis device.

The test result(s) can be transmitted to a user's smartphone and/or to a remote server.

### BRIEF DESCRIPTION OF THE DRAWINGS

Further features, details and advantages will become apparent from the detailed description below, and from an analysis of the attached drawings, in which:

FIG. 1 shows a schematic cross-section of a toilet equipped with a urine analysis device in the sense of the invention.

FIG. 2 represents schematically a detail of FIG. 1.

FIG. 3 illustrates a perspective view of a first case, according to a first embodiment, that can be implemented in the urine analysis device of FIG. 1.

FIG. 4 illustrates an exploded perspective view of the first case in FIG. 3 and a test assembly that can be implemented in the urine analysis device in FIG. 1.

FIG. 5 illustrates a partially exploded perspective view of a second case, according to a second embodiment, that can be implemented in the urine analysis device of FIG. 1.

FIG. 6 illustrates a detail of a third case, according to a third embodiment, that can be implemented in the urine analysis device of FIG. 1.

FIG. 7 shows another perspective view of the first case in FIG. 3.

FIG. 8 illustrates schematically a front view of a case according to a fourth embodiment, which can be implemented in the urine analysis device of FIG. 1.

FIG. 9 schematically shows the test assembly.

FIG. 10 illustrates schematically a flowchart according to an embodiment.

FIG. 11 illustrates schematically a flowchart according to another embodiment.

FIG. 12 schematically represents a detail of FIG. 1, according to another mode of implementation.

FIG. 13 shows a perspective view of a rear face of the device, according to another embodiment.

### DESCRIPTION OF THE METHODS OF IMPLEMENTATION

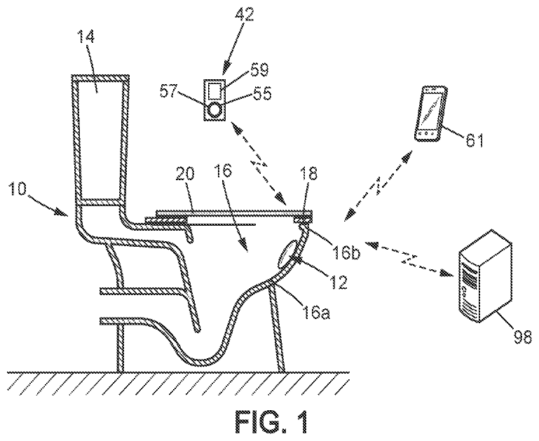

FIG. 1 illustrates a toilet 10 equipped with a urine analysis device 12. In a known manner, the toilet includes a water tank 14, a bowl 16, a seat 18 and a lid 20. The urine analysis device 12 is arranged on an inner wall 16a of the toilet bowl 16. Advantageously, the urine analysis device 12 is entirely received in the toilet bowl 16. Indeed, the urine analysis device 12 does not protrude from the upper rim 16b of the toilet bowl and thus remains unobtrusive.

Here, the urine analysis device 12 is positioned in the path of a urine stream secreted by a user. The urine analysis device receives a urine stream when a user urinates while sitting in the toilet. The position of the urine analysis device is then adapted for any type of user, male or female, regardless of age. The user can then urinate in the toilet without worrying about the position of the urine analysis device.

Here, the urine analysis device 12 is also positioned in the path of a flush from the tank 14. Thus, the urine analysis device can be flushed when the toilet is flushed. The urine analysis device is hygienic.

The urine analysis device 12 comprises a case 22 enclosing a test assembly 24. The test assembly is intended to analyze the urine being received in the urine analysis device.

The case 22 is removably arranged in the toilet bowl 16. The analysis device 12 can then be removed or repositioned in the toilet. The urine analysis device is discreet. In addition, the urine analysis device can be removed to recharge a battery 94 or a consumable 44 of the test assembly 24.

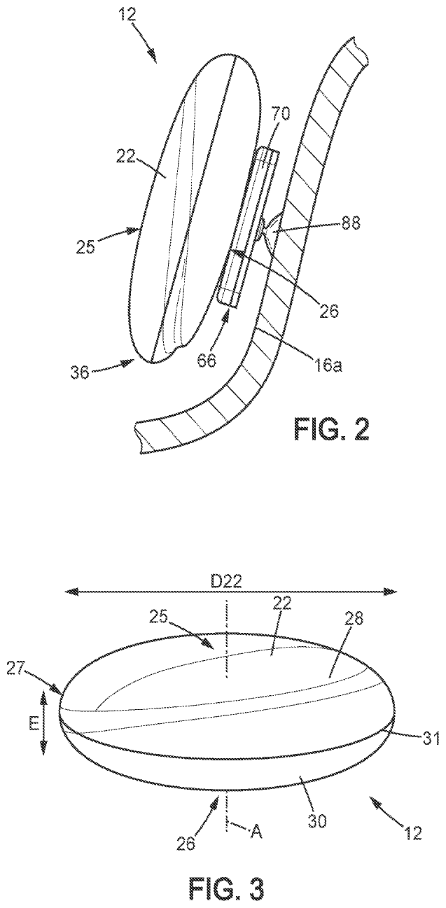

In the example shown in FIG. 2, the case 22 is arranged on the toilet wall 16a. The case is positioned by a fastener 66. The fastener 66 comprises a suction cup 88 intended to cooperate with the wall 16a of the toilet and magnets 70. The magnets 70 are intended to cooperate with magnets 23 (or pieces of ferromagnetic material) arranged inside the case. This configuration allows the case to be easily removed or repositioned in the toilet.

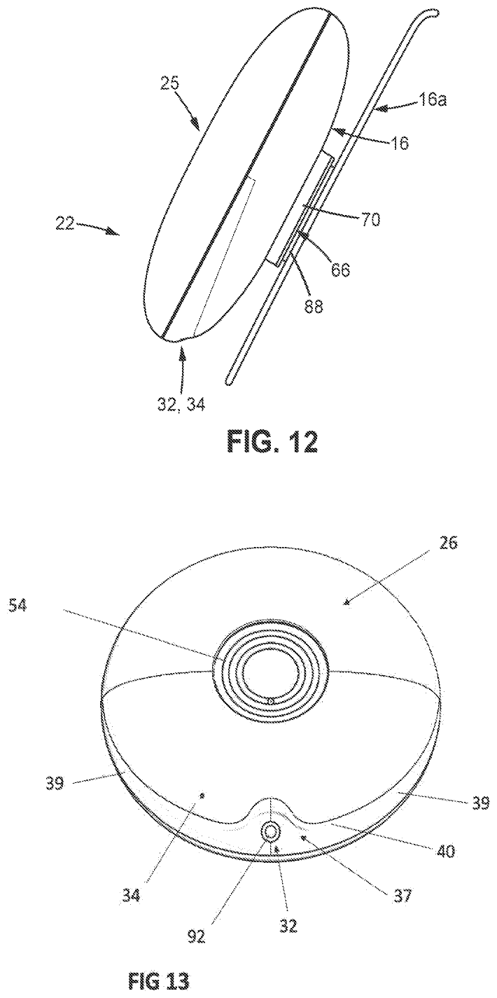

In the example shown in FIG. 12, the fastener 66 is further circular in shape. The fastener 66 fits into a complementary housing provided on the case 22. The housing may be provided at battery charging connectors 94 of the urine analysis device 12. Thus, the charging connectors can be protected from the water of the toilet 10.

Mechanical aids, including indentations, may be provided on the fastener 66. The mechanical aids may facilitate the positioning of the case 22 when inserted into the toilet 10. The proper positioning of the case 22 in the toilet 10 ensures the proper functioning of the urine analysis device 12.

The fastener 66 may also include a ball-and-socket connection. The ball-and-socket connection allows the case 22 to be oriented to increase the likelihood of contact with urine being received in the toilet bowl 16.

Alternatively, the fastener 66 may be a hook mounted on a rim 16b of the toilet bowl 16.

General Characteristics of the Case

The case 22 has an outer shape of a circular pebble. In other words, the case 22 has a flattened spheroid shape. An axis A is the centerline of the case. The case 22 has a front face 25 and a rear face 26, substantially normal to the axis A. Advantageously, the front face 25 may be substantially rotationally symmetrical, which gives a streamlined appearance to the device once installed. Thus, urine can be collected directly from the faces 25, 26 of the case. The case serves as a urine collector.

The front face 25 faces the interior of the toilet bowl 16. The front face 25 is then intended to receive urine when the user urinates while sitting on the toilet 10. The rear face 26 faces the inner wall 16a of the toilet bowl 16. The front face 25 and the rear face 26 are connected by curved edges 27. The respective curved edges 27 of the front face and the rear face meet flush at an equatorial junction area. Thus, the outer surface of the case 22, consisting of the front face 25, rear face 26 and curved edges 27, is defined by curved lines, and forms a generally convex object. The case 22 has no ridges. Urine can flow over the entire outer surface of the case 22 without coming off the case or forming air bubbles, which can compromise a urine analysis.

A ridge can be defined as an abrupt transition between two surfaces. A curved surface can be defined as a surface with a continuous derivative and a small second derivative.

The outer surface of the case 22 may further be white or light colored. The color of the outer surface may be similar to that of the toilet, increasing the discretion of the device.

Generally speaking, the case 22 has a diameter D22, measured in the direction normal to the axis A, of between 50 mm and 150 mm. Preferably, a diameter D22 between 80 mm and 120 mm can be chosen; in some embodiments, a diameter close to 100 mm can be chosen. The case 22 also has a thickness E, measured in the direction of the axis A, of between 15 mm and 50 mm, preferably in the vicinity of 30 mm. Thus, the case 22 is sufficiently compact to be completely received in the toilet bowl 16 of the toilet 10. The urine analysis device 12 is discrete. In addition, the case 22 is large enough to systematically come into contact with urine being received in the toilet bowl 16. The user can then urinate in the toilet without worrying about the urine analysis device, or alternatively aiming summarily.

In another aspect, in one embodiment, the case has a general form factor such that the E/D22 ratio is in the range [0.2-0.5] and preferably even in the range [0.3-0.4]; such proportions are reminiscent of a natural pebble, and gives the device a soothing appearance.

The outer surface of the case 22 is smooth. Thus, the urine stream contacting the case 22 clings to and spreads over the outer surfaces of the case. Preferably, the case 22 is made of a hydrophilic material. For example, the material of the case 22 may be one of: a ceramic, a polyamide (PA), a silicone or a hydrophilic polymer. The outer surface of the case 22 may also be treated with a hydrophilic surface treatment, for example AcuWet® from Aculon, a hydrophilic polymer, or Pebax® from Arkema.

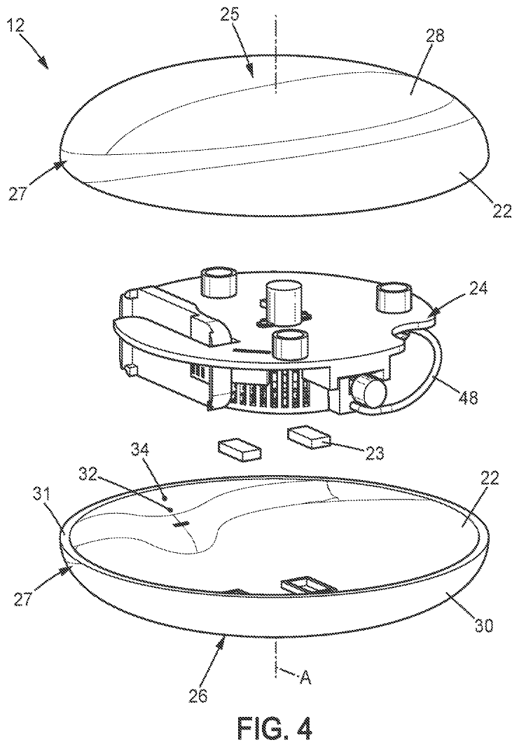

As more clearly seen in FIG. 4, according to a particular embodiment, the case 22 is formed as an assembly of two half-shells and consists of a front shell 28 and a rear shell 30. The front shell 28 and the rear shell 30 form an annular joint 31 (also referred to as edge 31) of the case 22, in a plane normal to the axis A. Assembly of the urine analysis device 12 is facilitated when the case 22 consists of the front shell 28 and the rear shell 30.

The front shell 28 and the rear shell 30 are joined together to hold the outer surface of the case 22 defined by curved lines. Then, the annular joint 31 between the front and rear shells allows urine to flow between the front face 25 and the rear face 26. The impact of the joint 31 on urine flow on the case is minimized.

The front shell 28 and the rear shell 30 can be assembled by screwing, gluing, clipping, magnetizing, or ultrasonic welding. Of course, other fastening means can be used to assemble the front shell 28 and the rear shell 30.

For example, the front shell 28 and the rear shell 30 are screwed together. Then, an internal portion of the front shell 28 comprises a thread. The thread of the front shell 28 is intended to cooperate with a complementary thread of the rear shell 30. This allows the case 22 to be easily disassembled for access to the test assembly 24 inside the case.

In another example, an internal portion of the rear shell 30 comprises a thread 140 intended to cooperate with a thread of the front shell 28. The two shells 28, 30 are assembled by screwing. Alternatively, the assembly of the two shells 28, 30 can be a bayonet system.

A seal may be present at the joint 31 between the front shell 28 and the rear shell 30. Thus, the housing 22 is waterproof. The interior of the case 22 is impervious to urine, water from the water tank 14 or bowl 16, and any other type of contaminant. Only the collection and drain ports connect the exterior and interior of the case, as described in more detail below.

Other Functions Supported by the Device

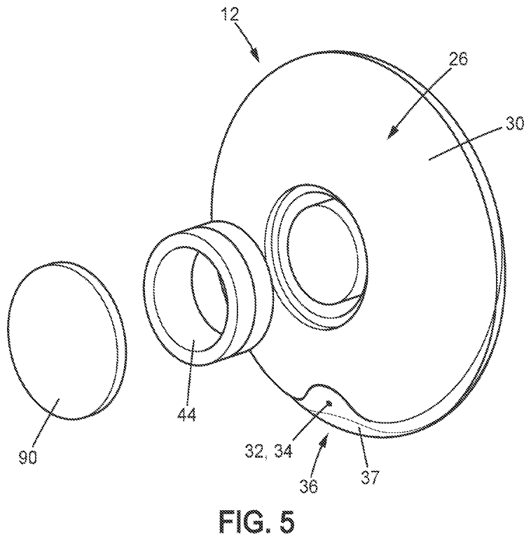

In the example shown in FIG. 5, a removable cover 90 is arranged on the rear shell. The removable cover allows for easy access to the test assembly 24. In particular, the removable cover allows the consumable 44 of the test assembly 24 to be refilled.

Here, the removable cover 90 is attached to the rear shell 30 by clipping, screwing or a bayonet mechanism. Of course, other means of attachment may be implemented to secure the removable cover to the rear shell 30. Alternatively, in another example, the removable cover could be attached to the front shell 28.

The removable cover 90 is arranged in a sealed manner. For example, a joint between the removable cover and the rear shell 30 may include a seal. Thus, the interior of the case 22 remains impervious to urine, water from the water tank 14 or bowl 16, and any other type of contaminant.

In another example, the removable cover 90 is formed by the front shell 28 of the case 22. The removable cover 90 can then be removed by unscrewing the front shell 28 from the rear shell 30. The case 22 has fewer joints that can be soiled and/or infiltrated by toilet water.

The case 22 has a collection port 32. The collection port can receive urine flowing by gravity on the outer surface of the case 22. Urine is collected directly from the sides 25, 26 of the case.

The collection port 32 is located on a lower end 36 of the case. The lower end 36 faces the bottom of the toilet bowl 16 when the case 22 is positioned in the toilet bowl. This position corresponds to a normal position of use. This position allows urine to be collected by gravity over the majority of the outer surface of the case.

In the present case, a distance D separating the collection port 32 from a lower edge 22a of the case 22 is less than 40 mm, preferably less than 20 mm. According to a particular embodiment, the collection port 32 is arranged a few millimeters above the bottom edge of the housing. Alternatively, the collection port could be on the bottom edge 22a.

The collection port 32 is an opening, typically circular, with a diameter preferably between 0.3 mm and 2 mm. The diameter of the collection port can be chosen to maximize the volume of urine collected from the outer surface of the case.

The case 22 has a drain port 34. The drain port allows the urine analysis device to be purged of excess urine.

The drain port 34 may be separate from the collection port 32. Then, the drain port is also located on the lower end of the case 22, in the vicinity of the collection port. The drain port is also a circular opening. The drain hole 34 has a diameter between 0.3 mm and 2 mm. In the normal position of use, as in an embodiment shown in FIG. 7, the drain port may be below the collection port.

The drain port 34 may also be located away from the collection port 32. The position of the drain port 34 may be chosen to facilitate access to the drain port by the test assembly 24. For example, FIG. 13 illustrates such a drain port 34. It will be described in more detail later.

Alternatively, as shown in FIG. 5, the drain port 34 may be the same as the collection port 32. A single port limits the number of openings to the interior of the case 22. Thus, the risk of introducing contaminants or elements that could clog the test assembly 24 is reduced.

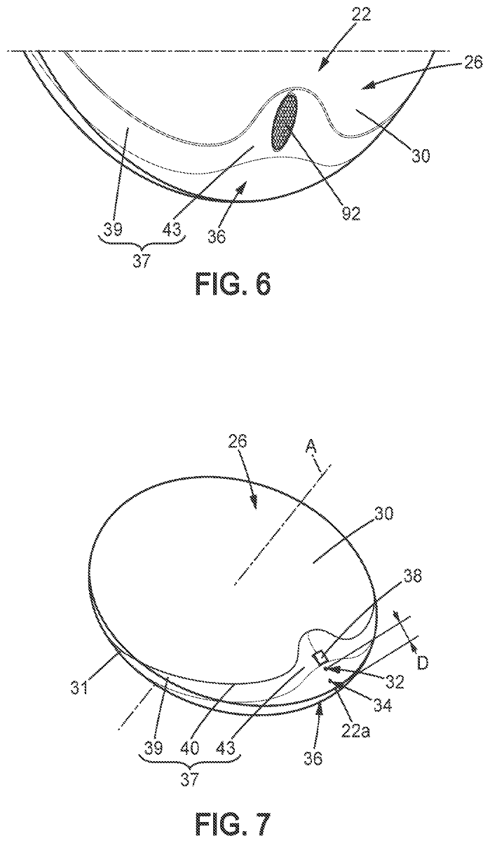

As seen in FIG. 6, the collection port 32 and the drain port 34 may be surmounted by a metal mesh filter 92. The filter covers the ports 32, 34. The mesh filter 92 is for example oblong in shape covering the ports 32, 34. The average mesh opening of the filter 92 is, for example, 20 microns. The filter 92 prevents the introduction of contaminants or elements that may clog the test assembly 24, and filters the urine received in the collection port 32. Alternatively, the filter 92 could be cleaned by an air stream generated by an air pump. In one embodiment, only the collection port 32 is protected by the wire mesh filter 92. The drain port 34, due to its arrangement, function and the flow rate it ejects, is not likely to contaminate the collection port 32.

In particular, in the examples shown in FIGS. 2 to 7, the collection port 32 and the drain port 34 are located on the rear face 26 of the case. Thus, the collection port and the drain port face the wall 16a of the toilet bowl 16 when the urine analysis device 12 is positioned in the toilet 10. This position allows the collection port and the drain port to be hidden from the user's view by the front face 25 of the case. The front face visible to the user resembles a simple, uniform pebble as already mentioned, without any singular points or holes, which gives the urine analysis device a somewhat magical appearance (it looks very simple, but produces very sophisticated results). Also, it should be noted that this position prevents the introduction of contaminants or elements that could obstruct the test assembly 24.

The collection port 32 and the drain port 34 are located in a recess 37. The recess 37 is formed on the case 22, in particular on the rear face 26. The recess extends inwardly from the case 22 for a maximum distance of between 1 and 4 cm, for example between 1.5 cm and 3 cm. The recess 37 has two lateral grooves 39 extending from the rear face 31 of the case to a central portion 43 of the recess 37 having the openings 32, 34. The depth of the lateral grooves 39, i.e., the distance from the rear face 26 to the interior of the case 22 in the direction of the axis A, increases from the rear face 31 to the central portion 43. Thus, the recess 37 forms a urine pathway from the front face 25 to the collection port 32. Advantageously, the recess 37 allows urine running down the front face 25 to be collected and directed to the collection port. Thus, the volume of urine reaching the collection port from the front face 25 of the case is sufficient for the needs of the analysis.

The central portion 43 of the recess 37, which functions as a buffer reservoir, typically extends inwardly from the case for a distance of between 1 and 4 cm, for example between 1.5 cm and 3 cm. Laterally, these same dimensions of the central portion 43 are applicable. In one embodiment, the central portion 43 of the recess has an area of at least 3 cm² and less than 8 cm². In this way, the recess 37 and in particular the central portion 43 of the recess 37 creates a buffer reservoir (open to the outside) that can receive urine that has run off the device, thereby ensuring that the collection port 32 receives urine.

The recess 37 may extend across the entire lower half of the device 12. Assuming that the device 12 has a spheroidal shape, it is possible to angularly define each point of the joint 31 about the axis A. By defining the collection port 32 as the angular zero, the location of the joint 31 from which the grooves 39 form can be at 90° (on both sides), or between 80 or 90°. Alternatively, as shown in FIG. 13 for example, said location may be closer to the collection port 32 and may be between 20° and 55°. As for the central portion 43, it is located at less than 20°. Thus, depending on said location of the starting point of the grooves 39 on the joint 31 (90°, 80°, 55° or 20°, etc.), the grooves 39 are more or less long.

In one embodiment shown in FIG. 13, for example, only the collection port 32 is in the recess 37. The drain port 34 is located on the rear face 26, outside the recess 37 (typically between 20 and 40°). In the normal position of use of the device 12, the drain port 34 is therefore higher than the collection port 32. The spacing between the two ports 32, 34 allows the recess 37 to be uncluttered. In addition, it has been observed that the drain port 34 so arranged does not generate contamination of the collection port 32.

A border 40 bounding the recess 37 is rounded. In other words, the border 40 is defined by curved surfaces. The rim 40 has no ridge. Urine in contact with the rear face 26 can run off to the collection port 32 without falling out of the case or forming air bubbles. Thus, the volume of urine reaching the collection port from the rear face of the case is increased. The absence of ridges is verified along the border 40 (along a line) and also orthogonally to the border 40 (also along lines). In other words, the three-dimensional surface defining the recess 37 (and including the border 40) does not have a ridge. This means that the transition from the rear face 30 to the border 40 is ridge-free and the transition from the border to the interior of the recess is ridge-free.

In one example embodiment, the collection port 32 is positioned 8 mm below the border 40.

The border 40 of the recess 37 may have an inflection between each lateral groove 39 and the central portion 43, such that, following the edge of the recess 37, the lateral groove 39 is convex (convexity is defined from the standpoint of the material of the device and not from the standpoint of the negative space complementary to the device), and then the inflection comes and the central portion 43 is concave, and then another inflection comes and the other lateral groove 39 is convex. Alternatively, the border 40 of the recess 37 has no inflection and remains convex.

For use purposes (i.e., the urine analysis device 12 is disposed laterally in the middle on the inner wall 16a of the bowl 16a), the recess 37 is typically symmetrical. However, the recess may have a slight asymmetry to favor installation slightly on a left or right side of the inner wall 16a of the bowl 16, and the collection port 32 may be slightly positioned on a right or left side (so that in use the collection port 32 is substantially centered laterally on the inner wall 16a of the bowl 16).

The distance between the border 40 and a lower edge of the device 12 (i.e., the distance of the segment intersecting the border 40 and the lower edge of the device, the segment being orthogonal to the tangent of said edge at the intersection) increases from the location of the joint 31 from which the lateral groove extends (i.e., the beginning of the lateral groove) to the central portion 43.

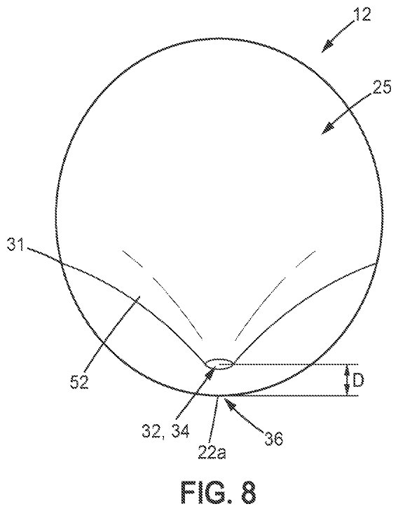

Alternatively, in the example shown in FIG. 8, the collection port 32, confounded with the drain port 34, is located on the front face 25 of the case. Thus, urine flowing down the front face reaches the collection port more directly.

As illustrated, the collection port 32 is on a projection 52 of the front face 25. The projection 52 extends from the joint 31 to the lower end 36 of the case having the port 32. The projection also forms a pathway for urine from the joint 31 of the case to the collection port 32. The protrusion allows urine dripping from the front face 25 to be collected and directed to the collection port.

It should be noted that the collection port may have an upwardly directed mouth, or a downwardly directed mouth, without excluding a purely radially directed mouth (i.e. flat to the general surface of the housing).

The case 22 is not limited to the only modes of realization described above with regard to the figures, but is, on the contrary, susceptible of numerous variants accessible to the man of the art.

In particular, the case 22 may take any geometric shape defined by curved lines. In particular, the case may have a diamond or inverted teardrop shape. In this case, the case has a point on the lower part to direct the urine towards the collection orifice 32.

The collection port 32 and the drain port 34, if any, may be located on a positive relief, such as a projection, or a negative relief, such as a gutter or recess. In general, the relief can be of any geometry that allows urine to flow over the case 22 and be directed to the collection port 32 without coming off the case or forming air bubbles.

In one embodiment, the collection port 32 is arranged on the front face 25, while the drain port 34 is located on the rear face 26.

Test Assembly

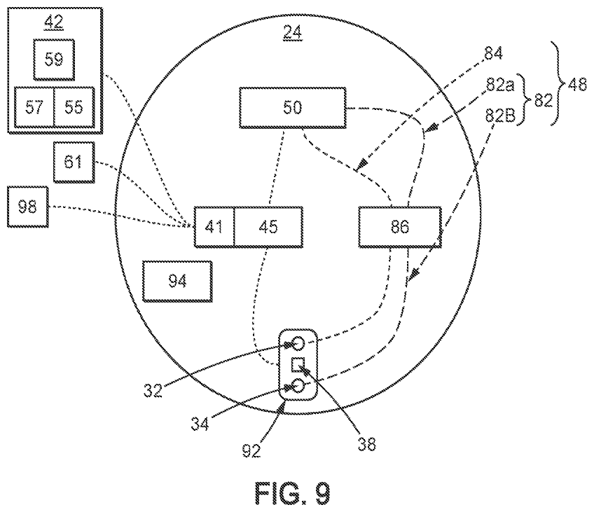

Subsequently, the test assembly 24 is described in more detail with reference to FIG. 9.

The test assembly 24 is controlled by an electronic control unit 45. The electronic control unit 45 is within the case 22. The electronic control unit 45 controls the components of the test assembly to perform a urine analysis and obtain one or more test results.

The test assembly 24 includes an analysis system 50. The analysis system 50 performs the analysis on urine collected from the collection port 32 to establish one or more results. Here, the analysis may be one or more of:
an analysis with colorimetric strips, conventional or of the lateral or vertical flow immunoassay type: urine is received on a strip containing one or more reagents sensitive to one or more components of the urine, and an analysis by optical detection is used to obtain an analysis result;
an analysis with a micro-fluidic chip, of the “lab on a chip” type: the chip mixes urine with one or more reagents contained in microdroplets, and an analysis by optical detection makes it possible to obtain an analysis result;
an analysis with a microfluidic chip on a porous support: the mixing of the urine with one or more reagents is obtained by capillary action;
an analysis with a field effect transistor, as described in the published patent application Withings EP3562407;
an analysis by a urine conductivity measurement;
an analysis by a pH measurement exploiting a metric pH probe;
an analysis by a spectrophotometric measurement;
an analysis by an electrochemical measurement.

The test assembly 24 comprises a urine delivery means 48. The urine delivery means 48 allows urine to be delivered from the collection port 32 to the drain port 34 through the analysis system 50. Preferably, the urine delivery means 48 includes a collection channel 82, a purge channel 84 and a pump 86.

Collection channel 82 connects collection port 32 to analysis system 50. The collection channel 82 allows urine to be conveyed from the collection port 32 to the analysis system 50.

The purge channel 84 connects the analysis system 50 to the drain port 34. In particular, the purge channel allows for the evacuation of urine contained in the urine delivery means 48. Preferably, the purge channel 84 is hydrophobic, allowing better evacuation of the urine. The risk of contamination of the urine conveyed between two successive collections is therefore reduced.

The pump 86 is arranged between a first portion 82a and a second portion 82b of the collection channel 82.

The pump 86 may draw urine from the collection port 32. For example, the pump 86 aspirates between 5 microliters and 1 mL, preferably about 20 microliters. Then, the pump can deliver a sufficient volume of urine to perform a conclusive analysis. A suction rate of the pump is chosen according to the diameter of the collection port. Thus, the pump can draw urine from the collection port to the analysis system 50 without forming air bubbles.

The pump 86 may also draw air from the collection port 32. The air flows through the urine delivery means 48 to the drain port 34 to discharge the urine contained in the urine delivery means. The urine collected for analysis is then protected from possible contamination by a previous collection.

Alternatively, the pump 86 could draw water upon activation of the toilet flush 10 to discharge urine contained in the urine delivery means 48.

Here, the pump 86 is a piezoelectric type pump. By using a piezoelectric pump, the urine analysis device can be free of controlled valves, which simplifies the urine analysis device.

Alternatively, the pump may be a pneumatic system. Then, the pneumatic system is configured to create a negative pressure to draw urine from the collection port, and a positive pressure to push urine to the analysis system 50 and the drain port. This solution allows for precise control of the volume collected by the urine analysis device 12.

The test assembly 24 includes a urine presence sensor 38. The urine presence sensor 38 is arranged in the vicinity of the collection port 32. The urine presence sensor then detects when urine is present in the vicinity of the collection port.

According to one embodiment, the urine presence sensor 38 could form a ring around the collection port. The integration of the urine presence sensor into the urine analysis device is then discrete.

Preferably, the urine presence sensor 38 is a temperature sensor, for example a thermistor. This is because the temperature sensor can distinguish between urine and water from the toilet 10. In addition, the temperature sensor can also be used to measure the temperature of the urine. In particular, the temperature of the urine can be used to detect periods of fertility. The use of a temperature sensor then reduces the number of components operated by the test assembly 24 to perform an analysis. The complexity and cost of manufacturing the urine analysis device is reduced.

Alternatively, the urine presence sensor 38 may be any type of liquid sensor, such as a capacitive or resistive type sensor. Then, a temperature sensor is separate from the urine presence sensor. The temperature sensor may be dedicated to measuring the temperature of the urine, in particular to detect a fertile period.

Communication and System Aspects

The urine analysis device includes a communication module 41. The communication module 41 is wireless. The communication module 41 operates a local area network, such as Bluetooth, Low-Energy Bluetooth (BLE), or Wi-fi. The local area network allows a battery 94 of the test assembly 24 to be preserved. Thus, the autonomy of the analysis device 12 is increased.

Alternatively, the communication module 41 may operate a cellular telecommunications network. The cellular telecommunication network may for example be GSM, 3G, 4G, 5G, 4G-LTE. The communication module 41 then has a longer range.

Alternatively still, the communication module 41 may operate a gateway connected to the cellular telecommunications network. In particular, the gateway may be a router, for example a Wi-fi router connected to the cellular network, a hub, i.e., a device connected directly to the cellular network, or the user's smartphone. Then, the urine analysis device 12 can be connected to the cellular network without compromising the battery life 94.

Preferably, the communication module 41 uses Bluetooth Low Energy (BLE) technology to communicate with a user's smartphone 61 and Wi-Fi technology to connect to a remote server 98.

The communication module 41 allows an analysis to be triggered via a remote control.

Preferably, the analysis is initiated from the user's smartphone 61. The user may initiate an analysis from an app on the smartphone. The user has control over the urine analysis device 12, and can choose when to perform an analysis. The user can also be identified through the use of the smartphone. The analysis performed can then be tailored to the user. The user can also select which test they wish to perform.

Alternatively, the analysis is initiated by communication with a remote device 42 in the vicinity of the toilet. The remote device has a button 55. The user can then press the button to initiate an analysis. The user has control over the urine analysis device, and can choose when to perform an analysis.

The button 55 can be provided with a biometric sensor 57. The button can then identify the user pressing the button. Then, an analysis relevant to the identified user can be performed by the urine analysis device. Furthermore, the analysis performed can be selected based on the identified user, and the results sent to enrich the history of that identified user.

The remote device 42 also includes a display 59. For example, the display may consist of one or more colored light emitting diodes (LEDs). The display may also include a screen. The display can inform the user. For example, the user may be informed that a button press has been perceived, and/or that the user has been identified and/or that an analysis is about to be performed.

Alternatively, the remote device 42 may be a connected bracelet associated with the user. In this case, the user may be automatically detected when in the vicinity of the toilet. The user can also be identified by the connected bracelet. Thus, an analysis can be launched automatically, without any action from the user. Note that the connected bracelet can be a connected watch.

The communication module 41 is also used to communicate the test result(s). The test result(s) may be one or more of: fertility, pregnancy, urinary tract infections, liver problems, kidney failure, uric acidosis, dehydration, heart disease and/or diabetes. The result(s) can also be an indicator of medication compliance.

The communication module 41 may send the test result(s) directly to the display 59 or the smartphone 61. The communication module may then be without a connection to the cellular telecommunications network. The test result(s) can be interpreted locally by the electronic control unit 45, or by the smartphone application. Thus, the operating costs of the urine analysis device 12 are reduced.

Alternatively, the communication module 41 may send the one or more results to a remote server 98. The remote server 98 may interpret the result(s). Operating a server reduces the computational capacity required locally to interpret the test result(s).

The remote server 98 may also be provided with storage capacity. Thus, the remote server may store the result(s) of a plurality of successive analyses.

The user can view and exploit one or more results, received directly from the analysis device 12 or from the server saying. For example, the user can view and exploit the results from the smartphone application. Alternatively, the user can access a website from a computer.

The case 22 may include one or more indicator lights (e.g., light emitting diodes, i.e., LEDs) arranged within the case opposite a translucent portion of the front face of the case so as to provide the user with visual feedback on the operation of the analysis device 12. Various colors and flashing patterns may be provided to indicate to the user various device statuses.

Finally, a button 192 may be accessible to the user from the case 22. The button 192 is accessible to the user to reset the urine analysis device 12. In particular, the button 192 may be accessible to the user when the removable cover 90 is removed.

Electronic Control Unit

Preferably, the control unit is built by a system-on-chip. A first Bluetooth Low Energy chip can take over the control of the electronic components of the urine analysis device. The first BLE chip may also support communication with the nearby device. A second Wifi chip can support the data exchange with the remote server. Thus, the second system-on-chip can be turned off when communication with the server is not in use. The power consumption of the control unit is improved, and the battery consumption is reduced.

The control unit can also be divided into different circuit boards. Each circuit board can control different electronic components of the urine analysis device. A main circuit can ensure the cooperation between the different circuit boards. Such a construction allows flexibility in the construction of the electronic control unit and its integration into the case.

The electronic control unit 45 is powered by the battery 94 provided inside the case 22. The battery 94 is of the lithium-ion type. The capacity of the battery is approximately 1080 mAh. Such a capacity makes it possible to ensure a satisfactory autonomy of the device without compromising the dimensions of the case.

The battery 94 comprises charging connectors 54. As particularly visible in FIG. 13, the charging connectors 54 are accessible from the exterior of the case 22 to allow recharging of the battery 94. For example, the case 22 may be placed on a stand to connect the charging connectors 54 to a power source. Alternatively, charging by induction could also be considered.

Process Aspects

Hereinafter, two processes for initiating a urine analysis and receiving the result(s) are described in more detail. The processes are implemented by the electronic control unit 45.

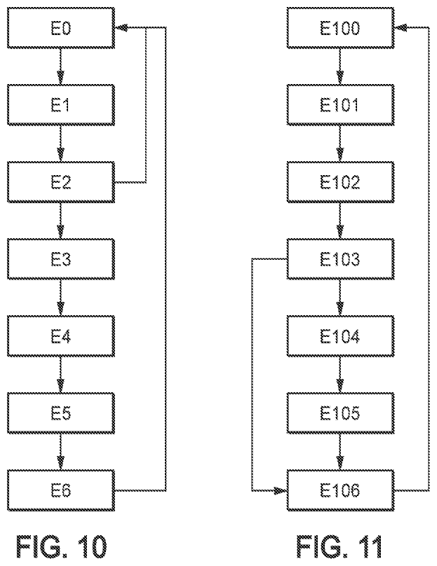

In the example shown in FIG. 10, the process is initiated by interaction with the user.

In step E0, the urine analysis device 12 is in an inactive state. In the inactive state, the urine analysis device is waiting to receive a request for analysis.

Then, step E1 corresponds to the reception of the analysis request. The analysis request can come from the user pressing the button 55. The analysis request can also be ordered from the smartphone 61. The analysis request may also be performed automatically, when the user is in the vicinity of the toilet 10. The display 59 may indicate to the user that their support has been received. The display may also indicate whether a user was recognized when the button was pressed.

In step E2, the urine analysis device 12 enters an active state. In the active state, the urine analysis device waits to detect urine near the collection port 32. The case 22 may further be equipped with LEDs to alert a user that the urine analysis device is in the active mode.

If no urine stream is received within a specified time period, then the urine analysis device 12 returns to the inactive state of step E0. If, instead, a urine stream is detected, the pump 86 is activated to deliver urine from the collection port 32 to the analysis system 50 (Step E3).

Step E4 involves performing an analysis on the collected urine. The analysis performed depends on the analysis device 50 in the case 22. The analysis performed may also depend on the user identified by the button 55. The analysis performed may also depend on the choice made by the user.

Step E5 corresponds to the transmission of the result(s). The result(s) can be transmitted directly to the user. The result(s) may also be sent to the server 98. The user may, for example, view and evaluate the result(s) on the smartphone application 61, or on a website. The result(s) may also be sent to a healthcare professional.

Finally, step E6 is to activate the pump 86 to purge the urine analysis device 12 of excess urine. The urine analysis device subsequently returns to the inactive state of step E0.

Alternatively, in the example shown in FIG. 11, the process is initiated when urine is detected on the case 22.

In step E100, the urine analysis device 12 is in an inactive state. Here, the inactive state corresponds to waiting for urine to be detected near the collection port 32.

Then step E101 is to detect the presence of urine in the vicinity of the collection port.

In step E102, the pump 86 is activated to draw urine from the collection port to the analysis system 50. The display 59 may indicate that an analysis is ready to be performed. Alternatively, the case 22, which has LEDs, may indicate that an analysis is ready to be performed.

In step E103, the urine analysis device 12 awaits receipt of an analysis request from a user. The analysis request may originate from the user pressing the button 55. The analysis request may also be commanded from the smartphone 61.

If the analysis request is not received within a specified time period, then the pump 86 is activated to purge the urine analysis device 12 of collected urine (Step E106). The urine analysis device returns to the inactive state of step E100.

If instead the analysis request is received, then an analysis is performed, at step E104. In addition, the display 59 may indicate to the user that their press was received. The display may also indicate whether a user was recognized when the button 55 was pressed.

Step E105 corresponds to the transmission of the result(s). The result(s) can be transmitted directly to the user. The result(s) may also be sent to the server 98. For example, the user may view the result(s) on the smartphone application 61, or on a website. The result(s) may also be sent to a healthcare professional.

Finally, step E106 is to activate the pump 86 to purge the urine analysis device 12 of excess urine. The urine analysis device subsequently returns to the inactive mode of step E100.

1. A urine analysis device comprising a case configured to be positioned entirely within a toilet,
the case having a front face for receiving a stream of urine directly from a user urinating on the toilet, a rear face opposite the front face, and a collection port, disposed on either the front face or the rear face, and
wherein the case contains a test assembly configured to perform an analysis on urine collected through the collection port.

2. The urine analysis device according to claim 1, wherein the case is ridge-free.

3. The urine analysis device according to claim 1, wherein the front face and/or the rear face comprises a relief forming a pathway for urine towards the collection port.

4. The urine analysis device according to claim 1, wherein the collection port is located in a recess.

5. The urine analysis device according to claim 4, wherein the recess extends towards a center of the case over a distance of between 1 and 4 cm.

6. The urine analysis device according to claim 4, wherein the recess comprises two lateral grooves extending from an edge of the case to a central portion of the recess, the collection port being in the central portion.

7. The urine analysis device according to claim 6, wherein the central portion has an area of between 3 and 8 cm².

8. The urine analysis device according to claim 6, wherein a depth of the two lateral grooves increases from the edge to the central portion.

9. The urine analysis device according to claim 6, wherein the recess has a border that has an inflection between at least one lateral groove of the two lateral grooves and the central portion.

10. The urine analysis device according to claim 9, wherein a distance between the border of the recess and the edge of the case increases from a beginning of the at least one lateral groove to the central portion.

11. The urine analysis device according to claim 1, wherein the collection port is located in the vicinity of a lower end of the case in a normal use position of the urine analysis device.

12. The urine analysis device according to claim 1, wherein the case has a generally circular pebble shape.

13. The urine analysis device according to claim 1, wherein the case comprises a drain port configured to drain urine.

14. The urine analysis device according to claim 1, wherein the case is made of hydrophilic material that is one of a ceramic, a polyamide, a silicone, and a hydrophilic polymer and/or the case is treated with a hydrophilic surface treatment.

15. The urine analysis device according to claim 1, wherein a diameter of the case is between 50 mm and 150 mm.

16. The urine analysis device according to claim 1, further comprising a urine presence sensor in a vicinity of the collection port, the urine presence sensor being configured to detect the presence of urine.

17. (canceled)

18. (canceled)

19. The urine analysis device according to claim 1, wherein the test assembly comprises one or more of: a plurality of test strips, a microfluidic chip, a field effect transistor, a conductivity measuring device, a pH measuring device, a spectroscopic measuring device, and an electrochemical measuring device.

20. A method of collecting urine using the urine analysis device according to claim 1, comprising:
detecting the presence of urine in the vicinity of the collection port,
collecting the urine flowing on the front face and/or the rear face,
analyzing the collected urine to establish one or several analysis results.

21. (canceled)

22. (canceled)

23. The urine analysis device according to claim 1, wherein the front face and the rear face are connected by curved edges.

24. The urine analysis device according to claim 4, wherein the recess is formed on the rear face.

25. The urine analysis device according to claim 15, wherein the diameter of the case is 100 mm.

26. The urine analysis device according to claim 16, wherein the urine presence sensor is a temperature sensor.
